(19) United States
(12) Patent Application Publication
(10) Pub. No.: US 2024/0382321 A1
(43) Pub. Date: Nov. 21, 2024

(54) CABLE TENSIONER FOR A WEARABLE ITEM

(71) Applicant: Open Bionics Ltd, Bristol (GB)

(72) Inventor: Steve Wood, Swindon Wiltshire (GB)

(73) Assignee: Open Bionics Ltd, Bristol (GB)

(21) Appl. No.: 18/661,046

(22) Filed: May 10, 2024

(30) Foreign Application Priority Data
May 17, 2023 (GB) 2307361.2

(51) Int. Cl. A61F 2/78; B65H 59/04

(52) U.S. Cl. CPC A61F 2/78; B65H 59/04; A61F 2002/7862

(57) ABSTRACT

A cable tensioner for a wearable item includes a rotatable dial, a rotatable reel that receives a cable wound therearound, a fixed portion, and a gear mechanism rotatably coupling the rotatable dial and the rotatable reel. The rotatable dial and rotatable reel are rotatable relative to the fixed portion. The rotatable dial is axially movable relative to the fixed portion between a first position, wherein the gear mechanism has a first gear ratio, and a second position, wherein the gear mechanism has a second gear ratio less than the first gear ratio. The first gear ratio has a value such that rotation of the rotatable dial permits fine adjustment of the rotatable reel and that the gear mechanism resistively holds a tension in the cable. The second gear ratio has a value that permits coarse adjustment of the reel and that the gear mechanism releases a tension in the cable.

### BACKGROUND OF THE INVENTION

This invention relates in general to a cable tensioner for a wearable item, and particularly, although not exclusively, relates to a cable tensioner for a wearable item with a gear mechanism.

A prosthesis is an artificial body part. EP3545914A1 discloses an example of a prosthetic limb. The prosthetic limb disclosed in EP3545914A1 uses a cable tensioning system to tighten a socket to the end of a user's residual limb and adjust the fit. However, previously-proposed cable tensioners apply tension in a series of gradations and are not capable of fine adjustment.

### SUMMARY OF THE INVENTION

According to a specific aspect, there is provided a cable tensioner, e.g., for a wearable item, the cable tensioner comprising: a rotatable dial; a rotatable reel configured to receive a cable and for the cable to be wound around the rotatable reel; a fixed portion, the rotatable dial and rotatable reel being rotatable relative to the fixed portion; and a gear mechanism rotatably coupling the rotatable dial and the rotatable reel.

The cable tensioner may be configurable between first and second configurations. For example, the rotatable dial may be movable, e.g. axially, relative to the fixed portion between a first position and a second position. The gear mechanism may be configured such that in the first position or configuration of the rotatable dial the gear mechanism may have a first gear ratio and in the second position or configuration of the rotatable dial the gear mechanism may have a second gear ratio. The first gear ratio may be greater than the second gear ratio.

The first gear ratio may have a value such that rotation of the rotatable dial may permit fine adjustment of the rotatable reel and/or that the gear mechanism resistively holds a tension in the cable. The second gear ratio may have a value that permits coarse adjustment of the reel and/or that the gear mechanism releases a tension in the cable.

The first gear ratio may be greater than the second gear ratio by at least an order of magnitude, e.g. 10 times greater. The first gear ratio may be approximately 10:1 (e.g., 10:1) or higher, for example 12:1. The second gear ratio may be approximately 1:1 (e.g., 1:1).

The gear mechanism may comprise an epicyclic gear arrangement. The epicyclic gear arrangement may comprise at least two gears mounted so that the center of one gear revolves around the center of the other.

The fixed portion may comprise a fixed gear. The fixed gear may correspond to a ring gear of the epicyclic gear arrangement. The fixed gear may be an internal gear, e.g., comprising teeth extending radially inward.

The reel may comprise a reel gear. The reel gear may correspond to a sun gear of the epicyclic gear arrangement. The reel gear may be an internal gear, e.g., comprising teeth extending radially inward. The reel gear may be axially spaced apart from the fixed gear (e.g., in the longitudinal direction).

The gear mechanism may comprise a dial gear. The dial gear may rotate with the rotatable dial. The dial gear may be coupled (e.g., rigidly connected) to the rotatable dial. The dial gear may be arranged to selectively engage the reel gear when the rotatable dial is in the second position. The dial gear may be an external gear, e.g., comprising teeth extending radially outward.

The gear mechanism may comprise at least one planet gear. The planet gear may be operatively disposed between the reel gear and the fixed gear. The planet gear may be an external gear, e.g., comprising teeth extending radially outward. The planet gear may be rotatably coupled to the rotatable dial. An axis of rotation of the planet gear relative to the rotatable dial may be eccentric relative to (e.g., spaced apart from) an axis of rotation of the rotatable dial relative to the fixed portion.

The dial gear may be fixedly attached at an end of a shaft. The shaft may be fixedly attached to the rotatable dial. The shaft may be eccentric relative to (e.g., spaced apart from) the axis of rotation of the dial relative to the fixed portion. The planet gear may rotate about the shaft.

The gear mechanism may comprise at least one further planet gear. The further planet gear may be axially spaced apart from the planet gear (e.g., in the longitudinal direction). The further planet gear may be connected to the planet gear such that they rotate together. The further planet gear may rotate about the shaft.

The planet gear may engage (e.g., mesh with) the fixed gear. The further planet gear may engage (e.g. mesh with) the reel gear when the rotatable dial is in the first position. The planet gear may not engage the fixed gear when the when the rotatable dial is in the second position.

The fixed portion may comprise a fixed gear. The reel may comprise a reel gear. The gear mechanism may comprise at least one planet gear and at least one further planet gear. The planet gear and further planet may be axially spaced apart and connected so as to rotate together. The further planet gear may engage the reel gear and the planet gear may selectively engage the fixed gear, e.g., when the rotatable dial is in the first position.

The fixed portion may substantially surround the reel, e.g., such that the reel rotates within the fixed portion. The fixed portion may rotatably support the reel. The fixed portion may comprise at least one opening for the cable to pass through and be wound round the reel.

One of the rotatable dial and the fixed portion may comprise at least one resiliently flexible part. The other of the rotatable dial and the fixed portion may comprise a ridge. The resiliently flexible part and the ridge may be configured to interact together and resiliently resist movement of the rotatable dial between the first and second positions.

The cable tensioner may comprise a base portion to which the fixed portion may be coupled. The base portion or fixed portion may be configured for coupling to the wearable item.

The wearable item may be a prosthesis (e.g., a prosthetic component, such as a limb or part thereof), an orthotic, an item of clothing (such as a shoe, boot or other garment), an item of sports apparel or any other wearable item.

According to another specific aspect, there is provided a wearable item comprising any of the above-mentioned cable tensioners and the cable, the cable tensioner and cable being arranged such that the cable tensioner tightens the cable to tighten the wearable item with respect to a wearer of the wearable item.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a perspective view of a cable tensioner according to an example of the present disclosure;

FIG. 2 is a side view of the cable tensioner according to the example of the present disclosure;

FIGS. 3a and 3b (collectively FIG. 3) are exploded perspective views of the cable tensioner according to the example of the present disclosure with FIG. 3a being a top perspective view and FIG. 3b being a bottom perspective view;

FIGS. 4a and 4b (collectively FIG. 4) are perspective sectional views of the cable tensioner according to the example of the present disclosure with FIG. 4a showing a rotatable dial of the cable tensioner in a first position and FIG. 4b showing the rotatable dial in a second position;

FIGS. 5a and 5b (collectively FIG. 5) are top sectional views of the cable tensioner according to the example of the present disclosure in the first position with FIG. 5a showing a showing a section through the teeth of a fixed gear and FIG. 5b showing a section through the teeth of a reel gear;

FIGS. 6a and 6b (collectively FIG. 6) are top sectional views of the cable tensioner according to the example of the present disclosure in the second position with FIG. 6a showing a showing a section through the teeth of the fixed gear and FIG. 6b showing a section through the teeth of the reel gear; and

FIG. 7 is a perspective of a wearable prosthetic item with the cable tensioner according to the example of the present disclosure.

### DETAILED DESCRIPTION OF THE PREFERRED EMBODIMENTS

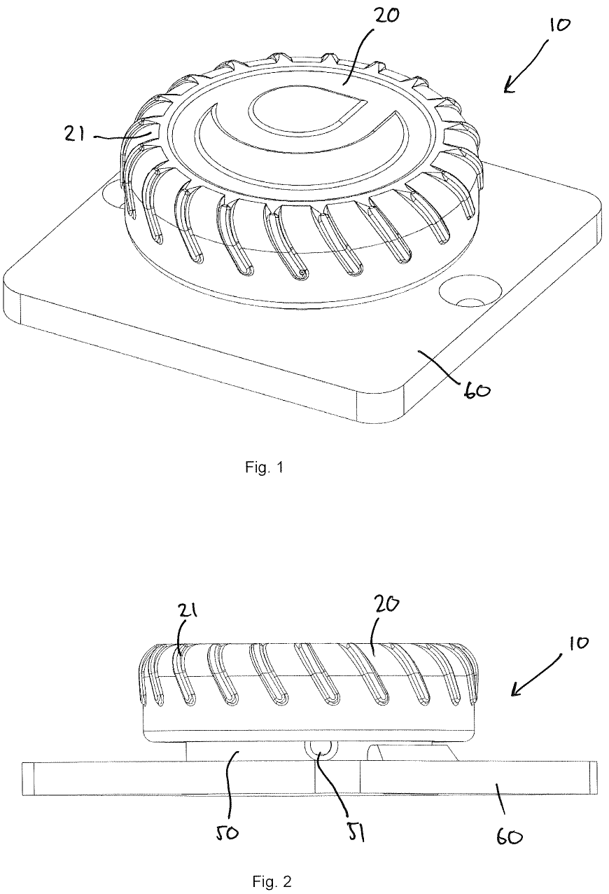

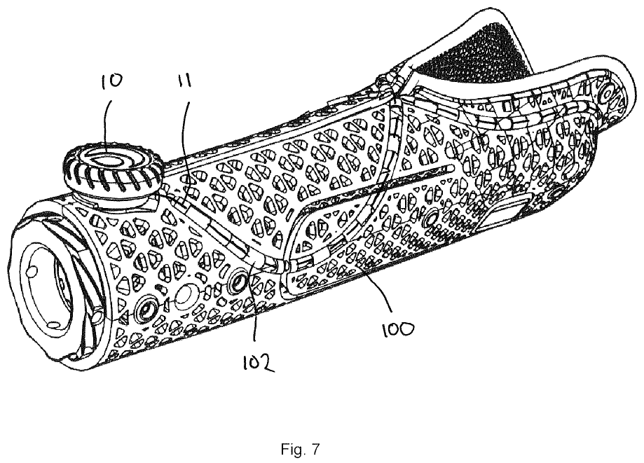

With reference to FIGS. 1 to 4 and 7, the present disclosure relates to a cable tensioner 10, which may be used for tensioning a cable 11 of a wearable item. In a particular example, as shown in FIG. 7, the wearable item may be a prosthesis 100, such as a prosthetic limb. However, in other examples (not shown), the wearable item may be an orthotic, an item of clothing (such as a shoe, boot or other garment), an item of sports apparel or any other wearable item. The cable 11 may be arranged such that a tension in the cable 11 may help secure the wearable item in place, e.g. in a manner similar to that of a lace in a shoe. The cable tensioner 10 may hold the tension in the cable 11.

The cable tensioner 10 comprises a rotatable dial 20, which a user (such as the wearer of the wearable item) may manually adjust, e.g. to adjust the tension in the cable 11. The dial 20 may be substantially tubular with a substantially circular cross-section. An end wall may close one end of the substantially tubular dial. As depicted, the dial 20 may comprise a plurality of grip features 21 (such as indentions or projections) that may improve the user's grip of the dial 20. The grip features 21 may be provided around a circumference of the dial 20.

The cable tensioner 10 further comprises a rotatable reel 30, which is configured to receive the cable and for the cable to be wound around the reel 30. The reel 30 may be substantially tubular with a substantially circular cross-section and may comprise a pair of rims 31a, 31b at opposite ends of the reel. The rims 31a, 31b may help constrain the cable to remain on the reel 30 as it is being wound. The reel 30 may further comprise one or more attachment points 32, to which the cable 11 may be secured.

The cable tensioner 10 further comprises a gear mechanism 40. The gear mechanism 40 may rotatably couple the dial 20 and the reel 30 together such that rotation of the dial 20 causes rotation of the reel 30. The gear mechanism 40 may comprise an epicyclic type gear arrangement. The epicyclic gear arrangement may comprise at least two gears mounted so that the center of one gear revolves around the center of the other. The gear mechanism 40 will be described in more detail below.

The cable tensioner 10 further comprises a fixed portion 50, which may rotatably support each of the dial 20 and reel 30. The dial 20 and reel 30 may each be rotatable relative to the fixed portion 50. For example, the fixed portion 50 may be substantially tubular with a substantially circular cross-section and may substantially surround the reel 30, e.g., such that the reel 30 rotates within the fixed portion 50. In a similar manner, the dial 20 may substantially surround the fixed portion 50, e.g., such that the dial 20 rotates around the fixed portion 50. The axis of rotation of the dial 20 may coincide with that of the reel 30. The dial 20, reel 30 and fixed portion 50 may be coaxial.

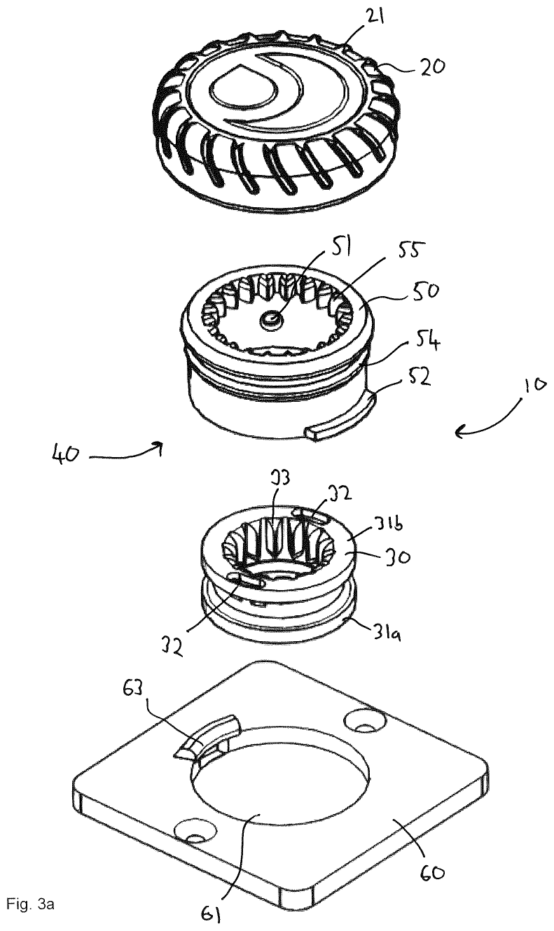

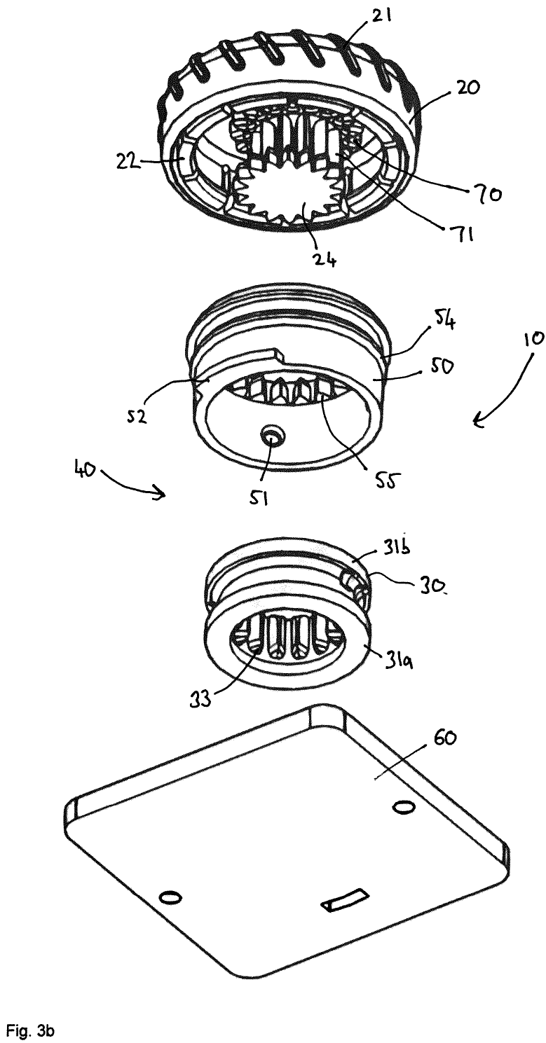

As best depicted in FIGS. 2 and 3, the fixed portion 50 may comprise at least one opening 51 for the cable to pass through and be wound round the reel 30. The dial 20 may not extend over the opening 51 so that the cable is free to extend beyond the cable tensioner 10.

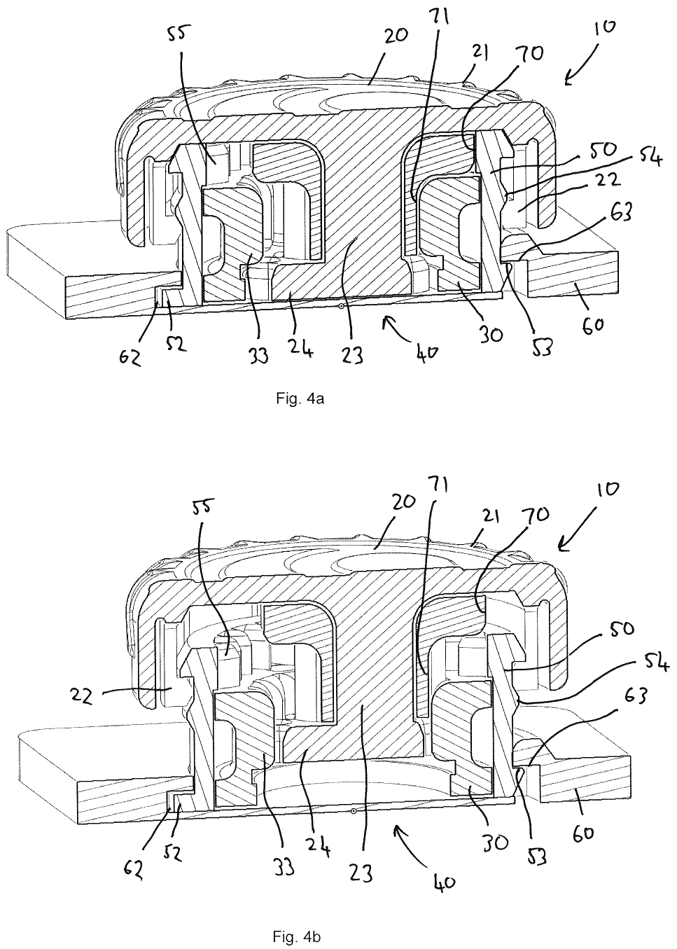

Referring still to FIGS. 1 to 4, the cable tensioner 10 may further comprise a base portion 60 to which the fixed portion 50 may be coupled. In the example shown, the base portion comprises a depression 61, which may receive the fixed portion 50. The depression 61 maybe substantially circular, e.g. to match an outer profile of the fixed portion 50. As best shown in FIG. 4, a tab 52 on a side of the fixed portion 50 may engage a corresponding recess 62, e.g. in a side wall of the depression 61. The fixed portion 50 may further comprise an abutment shoulder 53 that engages a corresponding abutment shoulder 63 on the base portion 60. The tab 52 and recess 62 may be diametrically opposed to the abutment shoulders 53, 63. During assembly the tab 52 may be first engaged in the recess 62 and the abutment shoulders 53, 63 may then ride over one another and provide a snap-fit as the fixed portion 50 is connected with the base portion 60. Once assembled, interactions between the abutment shoulders 53, 63 and between the tab 52 and recess 62 may prevent axial movement of the fixed portion 50 relative to the base portion 60. Further, an interaction between sides of the abutment shoulders 53, 63 and/or between the tab 52 and recess 62 may also prevent relative rotation of the fixed portion 50 and base portion 60. Although separate base portion 60 and fixed portion 50 have been described, it is also envisaged that the base portion 60 and fixed portion 50 may be integral with one another.

The base portion 60 may be configured for coupling to the wearable item. The base portion 60 may be flat or curved, e.g. to suit the shape of the wearable item to which it is to be attached. The cable tensioner 10 may be connected (e.g. via the base portion 60) to the wearable item by virtue of an adhesive, at least one fastener, stitching or any other coupling. For example, the base portion 60 may be sewn to a fabric or other material of the wearable item. The base portion 60 may also be integral with the wearable item. It is also envisaged that the base portion 60 may be omitted and the fixed portion 50 may couple directly to the wearable item, e.g. using similar means.

In addition to being rotatable about the fixed portion 50, the dial 20 may be axially movable relative to the fixed portion 50, i.e. in a longitudinal direction of the cable tensioner 10. FIG. 4a shows the dial 20 in a first axial position and FIG. 4b shows the dial in a second axial position. The dial 20 may axially slide relative to the fixed portion 50.

As is best shown in FIGS. 3b and 4, the dial 20 may comprise at least one resiliently flexible part. In the particular example shown, the dial 20 comprises a plurality of fingers 22 circumferentially distributed about the dial 20. Each finger 22 may form a cantilever extending in the axial direction and may resiliently flex. A distal end of each finger 22 may face and contact a surface of the fixed portion 50, thereby facilitating the sliding action in the axial and/or circumferential directions. The fixed portion 50 may comprise a ridge 54 circumferentially disposed about the surface of the fixed portion 50. The resiliently flexible fingers 22 and the ridge 54 may be configured to interact together and resiliently resist movement of the dial 20 between the first and second axial positions. For example, a projection at the distal end of each finger 22 may ride over the ridge 54 and cause the fingers 22 to flex. A force is therefore required by the user to move the dial 20 between the first and second axial positions. It is equally envisaged that the above-described arrangement may be reversed, e.g. with the fixed portion 50 comprising the resiliently flexible part(s) and the dial 20 comprising a corresponding ridge for the flexible part(s) to ride over.

The gear mechanism 40 may be configured such that in the first axial position of the dial 20, the gear mechanism may have a first gear ratio. The gear mechanism 40 may be further configured such that in the second axial position of the dial 20, the gear mechanism may have a second gear ratio. (It is also envisaged that the cable tensioner may be reconfigured in other ways to move between the first and second gear ratios, for example by virtue of a manually operated actuator that may act on the gear mechanism to change the gear ratio.) The first and second gear ratios may be different, for example the first gear ratio may be greater than the second gear ratio. In particular, more revolutions of the dial 20 may be required for a single turn of the reel 30 when the dial 20 is in the first axial position than when the dial 20 is in the second axial position.

With particular reference to FIGS. 4 to 6, the gear mechanism 40 will now be described in more detail. The fixed portion 50 may comprise a fixed gear 55. The fixed gear 55 may be an internal gear, e.g., comprising teeth extending radially inward. The fixed gear 55 may correspond to a ring gear of an epicyclic gear arrangement. In a similar manner, the reel 30 may comprise a reel gear 33. The reel gear 33 may be an internal gear, e.g., comprising teeth extending radially inward. The reel gear 33 may correspond to a sun gear of an epicyclic gear arrangement. The reel gear 33 may be axially spaced apart from the fixed gear 55 (e.g., in the longitudinal direction).

In addition to the reel gear 33 and fixed gear 55, the gear mechanism 40 may also comprise at least one planet gear 70. The planet gear 70 is arranged to selectively engage the fixed gear 55 when the dial 20 is in the first axial position. The planet gear 70 may be operatively disposed between the reel gear 33 and the fixed gear 55, e.g. in a gear train between the reel gear 33 and the fixed gear 55. The planet gear 70 may be an external gear, e.g., comprising teeth extending radially outward. The planet gear 70 may be rotatably coupled to the dial 20. An axis of rotation of the planet gear 70 relative to the dial 20 may be eccentric relative to (e.g., spaced apart from) an axis of rotation of the dial 20 relative to the fixed portion 50. In particular, the planet gear 70 may rotate about a shaft 23 and the shaft 23 may be fixedly attached to the dial 20. The shaft 23 may be eccentric relative to (e.g., spaced apart from) the axis of rotation of the dial relative to the fixed portion 50. In other words, the shaft 23 may not be coaxial with the dial 20. Accordingly, a center of the planet gear 70 revolves around a center of the dial 20.

The gear mechanism 40 may comprise at least one further planet gear 71. The further planet gear 71 may also be operatively disposed between the reel gear 33 and the fixed gear 55, e.g. in the gear train between the reel gear 33 and the fixed gear 55. The further planet gear 71 may be axially spaced apart from the planet gear 70 (e.g., in the longitudinal direction). The further planet gear 71 may be fixed to the planet gear 70 such that they rotate together, e.g. about the shaft 23. (The further planet gear 71 may simply be an axially spaced apart portion of the planet gear 70.) As such, the further planet gear 71 may also rotate eccentrically to the dial 20. The further planet gear 71 may be an external gear, e.g., comprising teeth extending radially outward. The further planet gear 71 may have fewer teeth that the planet gear 70.

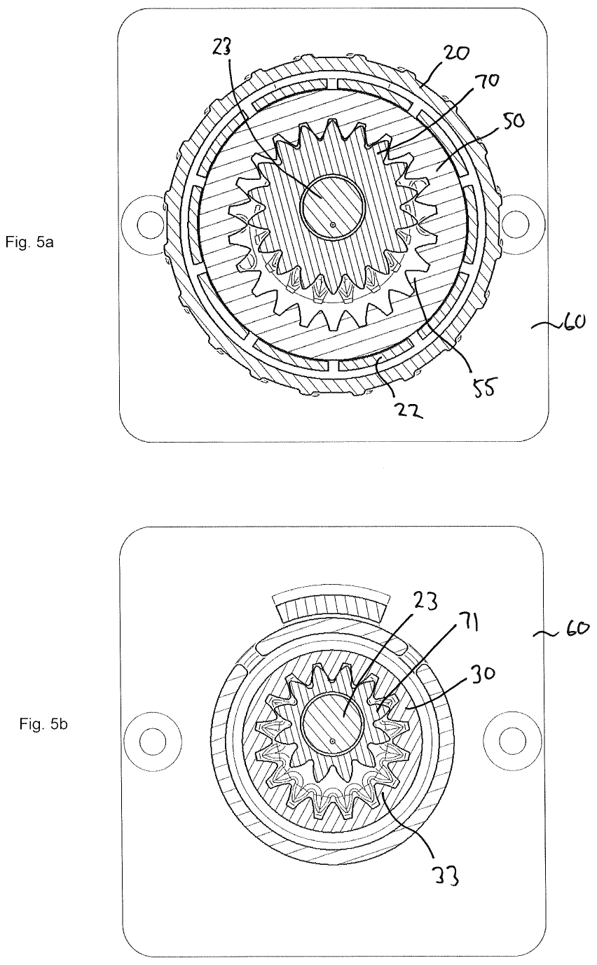

As shown in FIGS. 4a and 5a, when the dial 20 is in the first axial position, the planet gear 70 may engage (e.g., mesh with) the fixed gear 55. Rotation of the dial 20 causes the axis of rotation of the planet gears 70, 71 to rotate. As the planet gear 70 meshes with the fixed gear 55, this causes rotation of the planet gear 70 about the shaft 23. The further planet gear 71 also then rotates since it is fixed to the planet gear 70. As shown in FIGS. 4a and 5b, the further planet gear 71 may engage (e.g. mesh with) the reel gear 33 (when the rotatable dial is in the first position). Rotation of the further planet gear 71 (both about shaft 23 and the rotational axis of the dial 20) causes the reel 30 to rotate. This gear train occurs when the dial 20 is in the first axial position and defines the first gear ratio. The axial separation of the reel gear 33 and fixed gear 55 and the arrangement of the planet gears 70, 71 permits a compact gear mechanism 40 with a high gear ratio. The first gear ratio may be approximately 10:1 or higher, for example 12:1. (A gear ratio of 10:1 denotes that the dial 20 turns 10 times for each turn of the reel 30.)

The gear mechanism 40 may further comprise a dial gear 24. The dial gear 24 may be arranged to selectively engage the reel gear 33 when the dial 20 is in the second axial position. The dial gear 24 may be fixed relative to the dial 20, e.g. such that it rotates with the dial 20. The dial gear 24 may be an external gear, e.g., comprising teeth extending radially outward. In contrast to the planet gears 70, 71, a center of the dial gear 24 may be aligned with a center of the dial 20. In other words, the dial gear 24 may not be eccentric relative to the dial 20, reel 30 or fixed portion 50. As depicted, the dial gear 24 may be fixedly attached at an end of the shaft 23. The shaft 23 may not be coaxial with the dial gear 24 because the dial gear 24 is coaxial with the dial 20 and the shaft 23 is not coaxial with the dial 20. However, the dial gear 24 may provide a surface that holds the planet gears 70, 71 in place.

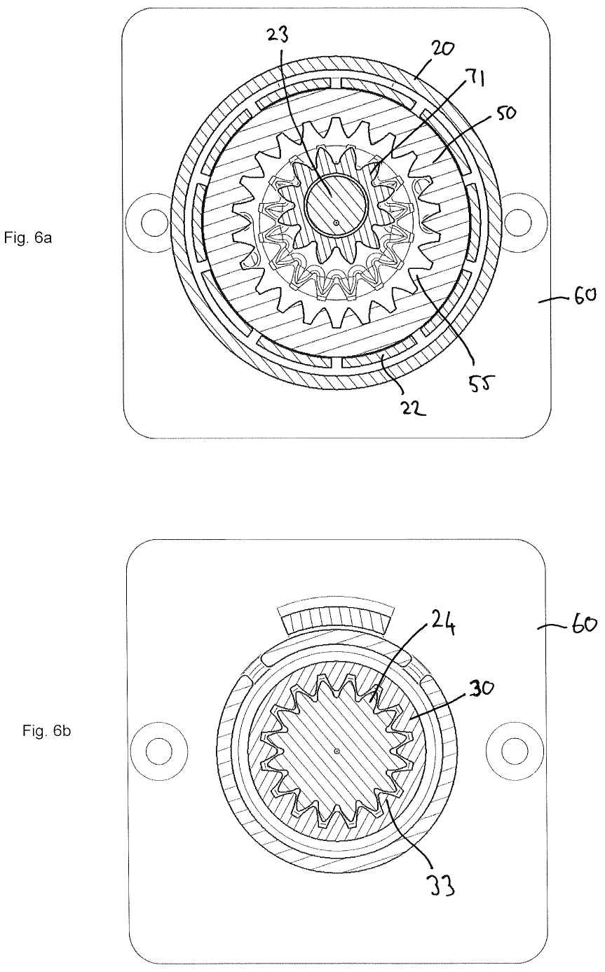

As shown in FIGS. 4b and 6, when the dial 20 is in the second axial position, the dial gear 24 may engage the reel gear 33. In the first axial position (depicted in FIG. 4a), the dial gear 24 is not in mesh with the reel gear 33. However, when the dial 20 is moved into the second position, the dial gear 24 moves into engagement with the reel gear 33 (as depicted in FIG. 6b). By contrast, as depicted in FIGS. 4b and 6a, the planet gear 70 moves out of engagement with the fixed gear 55 when the dial 20 is moved into the second position. The further planet gear 71 may or may not engage the reel gear 33 when the dial 20 is in the second position. The engagement of the further planet gear 71 with the reel gear 33 when the dial 20 is in the second axial position is immaterial because the planet gears 70, 71 are free to rotate due to the planet gear 70 not engaging the fixed gear 55.

As depicted in FIG. 6b, the dial gear 24 and reel gear 33 may be coaxial and may have a corresponding number of teeth. As such, the second gear ratio may be 1:1 such that the reel 30 turns in synchrony with the dial 20. In any event, the first gear ratio may be greater than the second gear ratio by at least an order of magnitude.

Although an epicyclic type gear mechanism 40 has been described above, it is also envisaged that other types of gear mechanism could be deployed. For example, the gear mechanism may comprise one or more worm gears or other gear combinations to achieve the desired gear ratios.

Components of the cable tensioner 10 may be 3D printed. In particular, the cable tensioner 10 may be 3D printed in a single printing process. However, the components may alternatively be manufactured separately (whether by 3D printing, molding or otherwise) and may then be assembled together.

With reference to FIG. 7, a wearable item, such as prosthesis 100, may comprise the cable tensioner 10. The cable 11 may be arranged so as to tighten an element of the wearable item. For example, the wearable item may define one or more pathways 102 for the cable 11 to extend through. One or two ends of the cable 11 may be connected to the reel 30 such that rotation of the reel may vary the length of the cable outside the cable tensioner 10. As such, rotation of the cable tensioner 10 tightens the cable 11 to apply a tension to the cable and tighten the wearable item with respect to a wearer of the wearable item.

In the first axial position of the dial 20, the first gear ratio may be such that rotation of the dial 20 permits fine adjustment of the reel 30 and thus the tension in the cable. The first gear ratio may also be such that the gear mechanism 40 may resistively holds the tension in the cable. In particular, a cable tension force required to turn the gear mechanism in reverse may be greater than a tension force required by the wearable item and that is likely to be encountered during tightening of the cable. The first gear ratio may be set such that the maximum desired cable tension for the wearable item is sufficient to overcome the resistance of the gear mechanism 40 when in the first axial position. This may prevent overtightening of the cable beyond a maximum desired cable tension.

In the second axial position of the dial 20, the second gear ratio may have a value that permits coarse adjustment of the reel and/or that the gear mechanism releases a tension in the cable.

In operation, the user may initially make coarse adjustments to the cable length with the dial 20 in the second position. They may then move the dial 20 into the first position to make fine adjustments to the cable length and tension. The large first gear ratio may then effectively lock the cable in place. When the user wants to release the tension in the cable, the user can move the dial 20 into the second position, at which point the gear ratio changes and the cable tension will be released.

The present invention advantageously provides a compact cable tensioner that allows fine adjustment of the cable length and tension (that is not limited to increase in increments). The present invention also provides an automatic means for locking the cable tension (e.g. without requiring a separate ratchet mechanism). Furthermore, the present invention is intuitive and ergonomic to use and may be readily operated one-handed.

Variations to the disclosed embodiments can be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A cable tensioner for a wearable item, the cable tensioner comprising:
a rotatable dial;
a rotatable reel configured to receive a cable and for the cable to be wound around the rotatable reel;
a fixed portion, the rotatable dial and rotatable reel being rotatable relative to the fixed portion; and
a gear mechanism rotatably coupling the rotatable dial and the rotatable reel, wherein the rotatable dial is axially movable relative to the fixed portion between a first position and a second position,
wherein the gear mechanism is configured such that in the first position of the rotatable dial the gear mechanism has a first gear ratio and in the second position of the rotatable dial the gear mechanism has a second gear ratio with the first gear ratio being greater than the second gear ratio, and
wherein the first gear ratio has a value such that rotation of the rotatable dial permits fine adjustment of the rotatable reel and that the gear mechanism resistively holds a tension in the cable and the second gear ratio has a value that permits coarse adjustment of the reel and that the gear mechanism releases a tension in the cable.

2. The cable tensioner of claim 1, wherein the first gear ratio is greater than the second gear ratio by at least an order of magnitude.

3. The cable tensioner of claim 1, wherein the first gear ratio is approximately 12:1, and/or the second gear ratio is approximately 1:1.

4. The cable tensioner of claim 1, wherein the gear mechanism comprises an epicyclic gear arrangement.

5. The cable tensioner of claim 1, wherein the fixed portion comprises a fixed gear.

6. The cable tensioner of claim 1, wherein the reel comprises a reel gear.

7. The cable tensioner of claim 6, wherein the gear mechanism comprises a dial gear, the dial gear being arranged to rotate with the rotatable dial and selectively engage the reel gear when the rotatable dial is in the second position.

8. The cable tensioner of claim 1, wherein the gear mechanism comprises at least one planet gear.

9. The cable tensioner of claim 8, wherein the planet gear is rotatably coupled to the rotatable dial and an axis of rotation of the planet gear relative to the rotatable dial is eccentric relative to an axis of rotation of the rotatable dial relative to the fixed portion.

10. The cable tensioner of claim 8, wherein the gear mechanism comprises at least one further planet gear, the further planet gear being axially spaced apart from the planet gear and rotating together with the planet gear.

11. The cable tensioner of claim 1, wherein the fixed portion comprises a fixed gear, the reel comprises a reel gear, and the gear mechanism comprises at least one planet gear and at least one further planet gear, wherein the planet gear and further planet are axially spaced apart and connected so as to rotate together, wherein the further planet gear engages the reel gear and the planet gear selectively engages the fixed gear when the rotatable dial is in the first position.

12. The cable tensioner of claim 1, wherein the fixed portion substantially surrounds the reel such that the reel rotates within the fixed portion.

13. The cable tensioner of claim 1, wherein one of the rotatable dial and the fixed portion comprise at least one resiliently flexible part and the other of the rotatable dial and the fixed portion comprise a ridge, the resiliently flexible part and ridge being configured to interact together and resiliently resist movement of the rotatable dial between the first and second positions.

14. The cable tensioner of claim 1, wherein the cable tensioner comprises a base portion to which the fixed portion is coupled, the base portion being configured for coupling to the wearable item.

15. A wearable item comprising the cable tensioner of claim 1 and the cable, the cable tensioner and cable being arranged such that the cable tensioner tightens the cable to tighten the wearable item with respect to a wearer of the wearable item.
